(19) United States
(12) Patent Application Publication
(10) Pub. No.: US 2008/0267940 A1
(43) Pub. Date: Oct. 30, 2008

(54) Methods of making concentrated fibrinogen containing compositions and associated systems for preparing fibrin glue

(76) Inventors: Syed F. Mohammed, Salt Lake City, UT (US); Sivaprasad Sukavaneshvar, Murray, UT (US)

Correspondence Address: THORPE NORTH & WESTERN, LLP., P.O. Box 1219, SANDY, UT, 84091-1219, US

(21) Appl. No.: 12/079,931

(22) Filed: Mar. 28, 2008

Related U.S. Application Data
(60) Provisional application No. 60/920,900, filed on Mar. 30, 2007.

(51) Int. Cl. A61K 38/43; C07K 14/75; C12N 9/00; A61K 33/06; A61P 7/00; A61K 38/36; A61K 35/58

(52) U.S. Cl. 424/94.1; 530/382; 435/183; 514/12; 424/542; 424/682

(57) ABSTRACT

The present invention is drawn to concentrated fibrinogen compositions and associate methods and use thereof. The concentrated fibrinogen compositions can be produced by adding a sufficient amount of a cationic agent, such as protamine, to a fibrinogen containing fluid to cause the fibrinogen to form a fibrinogen precipitate; collecting the fibrinogen precipitate by a collection means, such as centrifugation; and suspending or solubilizing the fibrinogen precipitate in a liquid vehicle to form a concentrated fibrinogen composition. The concentrated fibrinogen compositions can be incorporated into systems for making fibrin glues and be used in treating wounds.

This application claims the benefit of U.S. Provisional Patent Application Ser. No. 60/920,900, filed Mar. 30, 2007, which is herein incorporated by reference.

### BACKGROUND

Fibrin-based sealants are frequently used to reduce blood loss during/after surgery. The sealants, formed by mixing a concentrated solution of fibrinogen with thrombin and Ca²⁺ to produce fibrin, are applied to bleeding wounds and suture lines to help stop bleeding. Concentrated pooled human fibrinogen can be purchased, but it carries the risk of contamination or it is extensively processed to reduce that risk, which adds to the cost of commercial sealants. A method for producing concentrated fibrinogen from autologous blood on short notice would be an attractive alternative to relatively expensive commercial sealants.

The most common method for isolating fibrinogen from human blood is by cryoprecipitation to obtain fibrinogen concentrations of 20-40 mg/mL. This method requires several hours and results in a crude clotting factor concentrate that is useful to manage hemostatically-deficient patients, but is not practical for harvesting fibrinogen from small volumes of blood.

Fibrinogen can also be precipitated using chemical agents such as ethanol, polyethylene glycol (PEG), or ammonium sulfate. These methods require shorter time and provide fibrinogen concentrations ranging from 30 to >50 mg/mL. However, alcohol precipitation can cause elevated levels of ethanol in the fibrinogen concentrate, which can result in premature clotting of the fibrinogen and reduced factor XIII activity (and reduced sealant tensile strength). Isolation of fibrinogen with ammonium sulfate also precipitates a large amount of albumin, which can interfere with clotting. Precipitation of fibrinogen using PEG requires time-consuming preabsorption of prothrombin using BaSO₄ and MgSO₄, and the presence of PEG in the fibrinogen preparation is undesirable as it may render it less functional. Because of these limitations, chemical methods have not been pursued extensively for rapid harvesting of fibrinogen for clinical use as a sealant.

A commercial fibrinogen concentrate, Tisseel VH (Baxter Healthcare Corp., Westlake Village, Calif.), has been available in the United States since 1998. It is prepared by a complex process that includes isolation of fibrinogen from pooled human plasma and heat inactivation or solvent/detergent extraction to reduce the risk of viral contaminants. Tisseel is relatively expensive and has a somewhat limited shelf life.

As an alternative to commercial sealants, fibrin sealants have been prepared by mixing plasma or cryoprecipitate with bovine thrombin. However, as mentioned, sealants prepared with lower fibrinogen concentrations as in plasma may not possess desired physicochemical attributes and have limited ability to stop bleeding. Further, the preparation of cryoprecipitates is time-consuming and is generally not cost effective for small volumes. When the plasma or cryoprecipitate are obtained from blood banks, there is also an attendant risk of transmitting blood-borne pathogens.

Thus, there is a need for improved systems, methods, and compositions to make fibrin glue more readily and widely available.

### BRIEF DESCRIPTION OF THE DRAWINGS

FIG. 1 is a graphical representation of the recovery of fibrinogen in the f concentrated fibrinogen containing composition (percentage of fibrinogen in the original plasma) as a function of the protamine concentration used in the plasma. Data are shown as mean values±SD (n=4).

FIG. 2 is a graphical representation of the recovery of fibrinogen in the concentrate (percentage of fibrinogen in the original plasma) as a function of the precipitation temperature. Data are shown as mean values±SD (n=4).

FIG. 3 is a graphical representation of the Tensile strength (n=4) and adhesion strength (n=8) of a Fibrin glue or sealant as a function of calcium chloride concentration. A sealant fibrinogen concentration of 15 mg/mL was used. Data are shown as mean values±SD.

FIG. 4 is a graphical representation of the tensile strength as factor XIII (10 μg/mL) and calcium chloride (8.9 mM) were added to pure fibrinogen (15 mg/mL). Data are shown as mean values±SD (n=4).

FIG. 5 is a graphical representation of the tensile strength (n=4) and adhesion strength (n=8) of sealant as a function of cure time with or without calcium chloride (8.9 mM). The sealant was formed from precipitated plasma fibrinogen (15 mg/mL) at 37° C. and kept at 22° C. for 30 min. Data are shown as mean values±SD.

FIG. 6 is a graphical representation of the tensile strength (n=4) and adhesion strength (n=8) of sealant varied as a near-linear function of fibrinogen concentration and was greater with the addition of CaCl₂ (8.9 mM). Results from clotted Tisseel, plasma, and pure fibrinogen (15 mg/mL) are also shown. Data are shown as mean values±SD.

FIG. 7 is a graphical representation of the tensile strength of 15 mg/mL fibrinogen concentrates prepared from 1) pure fibrinogen, 2) pure fibrinogen precipitated with protamine, centrifuged, and then re-dissolved, and 3) plasma fibrinogen precipitated with protamine, centrifuged, and then re-dissolved. Data are shown as mean values±SD (n=4).

FIG. 8 is a graphical representation of the tensile and adhesion strengths of clots prepared from sealant in the presence of antifibrinolytic agents with and without calcium chloride (8.9 mM). A sealant fibrinogen concentration of 15 mg/mL was used. Antifibrinolytic agents used were aprotinin (3000 KIU/mL) and ε-aminocaproic acid (ε-ACA, 10 mg/mL). Data are shown as mean values±SD (n=4).

FIG. 9 is a schematic representation of a filter design for concentrating fibrinogen from whole blood. The filtration chamber can be designed for a range of blood volumes (e.g. 10-20 ml, 25-50 ml, 50-75 ml, 75-100 ml). The time from adding the blood to the mixing chamber to the recovery of concentrate is usually less than 15 min. The fibrinogen concentrate prepared from whole blood exhibits physicochemical characteristics similar to the commercially available fibrin glue Tisseel V (Baxter Healthcare, Calif.)

### DETAILED DESCRIPTION

Reference will now be made to the exemplary embodiments and specific language will be used herein to describe the same. It will nevertheless be understood that no limitation of the scope of the invention is thereby intended. Alterations and further modifications of the inventive features illustrated herein, and additional applications of the principles of the inventions as illustrated herein, which would occur to one skilled in the relevant art and having possession of this disclosure, are to be considered within the scope of the invention. It is also to be understood that this invention is not limited to the particular configurations, process steps and materials disclosed herein as these may vary to some degree. Further, it is to be understood that the terminology used herein is used for the purpose of describing particular embodiments only, and is not intended to be limiting as the scope of the present invention.

It is noted that, as used in this specification and the appended claims, singular forms of “a,” “an,” and “the” include plural referents unless the content clearly dictates otherwise.

As used herein, the term “active bleeding” refers to any loss of blood from the circulatory system, regardless of cause.

As used herein, the term “wound” or refers to any damage to any tissue of a subject. The wound may, but does not have to be experiencing active bleeding. The damage can be injury or surgically created and can be internal or external on the body of the subject. Non-limiting examples of injuries include ulcers, broken bones, puncture wounds, cuts, scrapes, lacerations, surgical incisions, and the like.

As used herein, “fluid” refers to a flowable composition and can include liquid, suspended solids, or other flowable masses. Fluids can be in the form of suspensions, emulsions, solutions, mixtures, colloids, or the like.

As used herein, the term “fibrinogen containing fluid” refers to any fluid, either biological or artificial, which contains fibrinogen. Non-limiting examples of such fluids include various forms blood plasma.

A “concentrated fibrinogen composition” refers to a fibrinogen composition derived from a fibrinogen containing fluid, the fibrinogen being present in a medium or liquid that is distinct compared to that of the fibrinogen containing fluid from which the concentrated fibrinogen is derived. The concentrated fibrinogen composition may, but is not required to, have a concentration which is greater than the concentration of the fibrinogen containing fluid. For example, a concentrated fibrinogen composition can have a fibrinogen concentration which is less than or equivalent to the concentration of fibrinogen in the original fibrinogen containing liquid, or can be at a concentration which is greater than the fibrinogen concentration of the original fibrinogen containing liquid. In other words, the term “concentrated” does not infer fibrinogen concentrations as they relate to the original fibrinogen containing fluid from which the concentrated fibrinogen composition is derived, only that it is concentrated enough to form a clot under appropriate conditions.

As used herein, the term “collecting” or “collection” when use with respect fibrinogen precipitate refers to the separation of the fibrinogen precipitate from the bulk of the fibrinogen containing fluid. Such a step does not require, but does allow for, actual gathering of the precipitate. The collection may occur through any number of means in the art including, but not limited to gravity separation, decanting, centrifugation, filtration, and the like.

As used herein, fibrinogen and clotting Factor I are synonymous

As used herein, the term “clotting agent” refers to any fluid or material that facilitates or causes clotting of fibrinogen-containing compositions to form a fibrin glue or sealant. Materials like calcium (e.g., calcium salt), magnesium (e.g. magnesium salt), thromboplastin, actin, thrombin, collagen, platelet suspension, precipitated or denatured proteins, complex carbohydrates, silica, zinc, diatomaceous earth, kaolin, Russel's viper venom, ristocetin, and mixtures thereof, are exemplary. However, clotting agent can also be found in the fluid typically present at a normal wound site, thereby causing the fibrinogen to form a fibrin glue or sealant, though typically at a slower rate.

As used herein, the term “cationic agent” refers to cationic materials that react or interact with fibrinogen to cause some amount of precipitation or flocculation, so that the precipitate or flocculent is separable from its fluid to at least some degree. Examples of appropriate cationic agents include amines such as protamine, polylysine, polyallylamine, histones, and mixtures thereof.

As used herein, the term “about” is used to provide flexibility to a numerical range endpoint by providing that a given value may be “a little above” or “a little below” the endpoint. The degree of flexibility of this term can be dictated by the particular variable and would be within the knowledge of those skilled in the art to determine based on experience and the associated description herein.

As used herein, a plurality of components may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0” should be interpreted to include not only the explicitly recited values of about 0.01 to about 2.0, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. This same principle applies to ranges reciting only one numerical value. Furthermore, such an interpretation should apply regardless of the breadth of the range or the characteristics being described.

With these definitions in mind, it has been recognized that it would be advantageous to provide a method for making a concentrated fibrinogen composition for use in reducing or stopping bleeding by causing the blood to clot. As such, the present invention provides for concentrated fibrinogen compositions, systems, and related methods of manufacture and use.

The present disclosure provides a method of making and clotting a concentrated fibrinogen composition. Steps can include adding a sufficient amount of a cationic agent to a fibrinogen containing fluid to cause the fibrinogen to form a fibrinogen precipitate, and collecting the fibrinogen precipitate, and after collecting, suspending or solubilizing the fibrinogen precipitate in a liquid vehicle to form a concentrated fibrinogen composition. An additional step includes clotting the concentrated fibrinogen composition.

In another embodiment, a method of treating wounds can comprise adding a sufficient amount of a cationic agent to a fibrinogen containing fluid to cause the fibrinogen to form a fibrinogen precipitate, collecting the fibrinogen precipitate, and after collecting, suspending or solubilizing the fibrinogen precipitate in a liquid vehicle to form a concentrated fibrinogen composition. Additional steps can include mixing the concentrated fibrinogen composition with a clotting agent to form a fibrin sealant, and applying an amount of the fibrin sealant to a wound, thereby forming a clot.

In another embodiment, a method of making a concentrated fibrinogen composition from blood can comprise adding a sufficient amount of a cationic agent to blood so as to cause fibrinogen present in the whole blood to form a fibrinogen precipitate, collecting the fibrinogen precipitate, and after collecting, suspending or solubilizing the fibrinogen precipitate in a liquid vehicle to form a concentrated fibrinogen composition.

In still another embodiment, a method of making and using an autologous fibrin glue can comprise collecting a fibrinogen containing fluid from a subject, adding a sufficient amount of a cationic agent to the fibrinogen containing fluid sample to cause the fibrinogen to form a fibrinogen precipitate, and collecting the fibrinogen precipitate. Additional steps include suspending or solubilizing the fibrinogen precipitate in a liquid vehicle to form a concentrated fibrinogen composition, and applying the concentrated fibrinogen composition to a wound of the subject to form a fibrin glue, wherein the fibrin glue forms a clot.

In another embodiment, a system for making a fibrin glue can comprise a first component being a fluid including 10 mg/ml to 200 mg/ml fibrinogen and at least one clotting factor selected from the group consisting of Factor II, Factor IX, Factor X, and Factor XIII. The system can further comprise a second component including a clotting agent for said fibrinogen. When the first component and second component are contacted, a fibrin glue can be formed.

In another embodiment, a method of making a concentrated fibrinogen composition can comprise adding a sufficient amount of protamine to a fibrinogen containing fluid to cause the fibrinogen to form a fibrinogen precipitate, collecting the fibrinogen precipitate by centrifugation, and after collecting, suspending or solubilizing the fibrinogen precipitate in a sodium citrate containing liquid vehicle to form a concentrated fibrinogen composition. The concentration of the fibrinogen in the concentrated fibrinogen composition can be at least twice the concentration of fibrinogen in the fibrinogen containing fluid.

In another embodiment, a system for making a fibrin glue can comprise a fluid including 10 mg/ml to 200 mg/ml fibrinogen and at least one clotting factor selected from the group consisting of Factor II, Factor IX, Factor X, and Factor XIII. When applied to a wound, a fibrin glue can be formed.

It is noted that when discussing the concentrated fibrinogen compositions, their methods of making, and related methods of use in fibrin glues and fibrin glue systems, each of these discussions can be considered applicable to each of these embodiments, whether or not they are explicitly discussed in the context of that embodiment. Thus, for example, in discussing concentrated fibrinogen composition for use in making fibrin glue, the concentrated fibrinogen composition can also be used in a system for making fibrin glue, and vice versa.

In accordance with the methods and systems of the present invention, fibrinogen can be collected from a variety of physiological and artificial fibrinogen containing fluids. In one aspect of the invention, the fibrinogen containing fluid can be whole blood. In another aspect, the fibrinogen containing fluid can be plasma, including typical plasma, as well as platelet rich plasma (PRR) and platelet poor plasma (PPP). The source of the blood or plasma can be a human source or other animal source. The present invention is particularly useful when it is desired the fibrinogen source of the fibrinogen containing fluid is also the target for use of the concentrated fibrinogen or, ultimately, the fibrin glue made from the concentrated fibrinogen. For example, when prepared in anticipation of surgery. Plasma fibrinogen levels vary considerably between individuals, being affected by age, sex, race, alcohol intake, and smoking, as well as certain diseases. Fibrinogen concentrations of 2-6 mg/mL are typical in normal patient populations; however, clots prepared from unconcentrated fibrinogen solutions can fail to provide desired mechanical properties, thereby leading to poor reproducibility and questionable efficacy as a sealant. The present disclosure provides for the ability to control fibrinogen concentration in the final concentrate, which in turn helps to minimize the variation in sealant performance.

Once a fibrinogen containing fluid is chosen, a cationic agent can be added to the fluid to cause the fibrinogen to precipitate or flocculate. There are a variety of cationic agents which can be used including various amines including protamine, polylysine, polyallylamine, histones, and mixtures thereof. In one embodiment, protamine is the cationic agent. Fibrinogen precipitation by a cationic agent, such as protamine, is rapid, and often results in much if not substantially all of the fibrinogen in the fibrinogen containing fluid being recovered. It also has the benefit of precipitating certain clotting factors, including Factor X, Factor XIII, and/or Factor II.

Once precipitated, the precipitated fibrinogen can be collected by any collection means known in the art, including but not limited to gravity settling, centrifugation, filtration, or combinations thereof. In one embodiment, the collection is accomplished by filtration. Filtration can be advantageous because it can be done using portable filtration devices, such as shown in U.S. Patent Publication No. 20070037132, which is incorporated herein by reference. In another embodiment, the collection of the precipitated fibrinogen can be accomplished by centrifugation.

Once collected, the fibrinogen precipitate can be suspended or solubilized in a liquid vehicle to form a concentrated fibrinogen composition. The liquid vehicle can be aqueous or non-aqueous so long as it is physiologically acceptable and does not significantly degrade or denature the fibrinogen. Examples of liquid vehicles include but are not limited to aqueous solutions of sodium citrate, sodium hydroxide, potassium hydroxide, heparin, heparan sulfate, other anionic solutions, mixtures thereof and the like. In one embodiment, the liquid vehicle is an aqueous sodium citrate solution.

The concentrated fibrinogen compositions of the present invention can have fibrinogen concentrations which are at least twice the concentration of the fibrinogen containing liquid from which the fibrinogen is derived. In other words, the methods of the present invention provide for at least a 100% increase in the fibrinogen concentration from the original fibrinogen containing fluid to the concentrated fibrinogen composition. In one embodiment, the fibrinogen can be present in the concentrated fibrinogen composition at a concentration of 10 mg/ml to 200 mg/ml. In another embodiment, the fibrinogen can be present in the concentrated fibrinogen composition at a concentration of 20 mg/ml to 100 mg/ml. In another embodiment, the fibrinogen can be present in the concentrated fibrinogen composition at a concentration of 20 mg/ml to 60 mg/ml. In a further embodiment, the fibrinogen can be present in the concentrated fibrinogen composition is least about 15 mg/ml.

An additional benefit of the above described methods of harvesting fibrinogen can be the simultaneous harvesting of the clotting factors which may be present in the fibrinogen containing fluid. Such clotting factors can include, but are not limited to, Factor X, Factor IX, Factor XIII, Factor II, Factor VIII, and the like, which are present in the plasma and whole blood. As such, in one embodiment, the concentrated fibrinogen compositions obtained by any of the above described methods can include at least one of Factor IX, Factor X, Factor XIII, Factor II, and Factor VIII. In another embodiment, the concentrated fibrinogen compositions obtained by the above described method can include at least two of Factor X, Factor IX, Factor XIII, Factor II, and Factor VIII. In yet another embodiment, the concentrated fibrinogen compositions obtained by any of the above described methods can include each of Factor X, Factor IX, Factor XIII, and Factor VIII. When the concentrated fibrinogen containing composition is derived from whole blood or plasma, the at least one clotting factor, e.g. Factor X, Factor II, Factor IX, or Factor XIII, can be present in the concentrated fibrinogen composition at a concentration which is at lease twice the concentration of the clotting factor in the plasma or whole blood, though this is not required. The mere presence of these clotting factors in the concentrated fibrinogen composition can provide a benefit for enhancing clotting function.

The concentrated fibrinogen compositions prepared by any of the methods of the present invention can be used to prepare a fibrin sealant or glue which can be applied to wounds. Examples of wounds include accidental cuts, punctures, internal bleeding, other injuries, surgical incisions, and the like. Thus, by “wound,” this term does not necessarily imply that the wound is open to the atmosphere, but rather, it is open compared to its normal state. Typically, wounds will be open to the atmosphere, but internal bleeding is also included herein. The fibrinogen compositions of the present disclosure can be applied to wounds by mixing the concentrated fibrinogen composition with an amount of thrombin or other clotting agent in order to form the fibrin sealant. The fibrin sealant can be applied to the wound quickly forming a clot which reduces or eliminates active bleeding from the wound. In one example, if thrombin is used, it can be present in the fibrin sealant in amounts from 50 units/ml to 500 units/ml of the fibrin sealant.

The fibrin sealants of the present invention can also include other compounds which can aid in wound healing and blood clotting, such as any of the clotting factors (discussed above) or clotting agents. In one embodiment, the fibrin sealant can include at least one clotting factor selected from the group of Factor X, Factor XIII, Factor II, Factor VIII and mixtures thereof. When present, the Factor VIII can aid in forming a more viscous sealant with desirable attributes. One benefit of having Factor XIII included in the fibrin sealant is that it ensures that the fibrin sealant is cross-linked and, therefore, less susceptible to fibrinolysis. Factor XIII requires calcium as a cofactor to crosslink fibrin, increase the tensile strength of clots, and diminish their breakdown.

Clotting agents which can be used in the fibrin sealants or glues in combination with the concentrated fibrinogen composition include, but are not limited to, calcium salts, magnesium salts, thromboplastin, actin, thrombin, collagen, platelet suspension, precipitated or denatured proteins, complex carbohydrates, silica, zinc, diatomaceous earth, kaolin, Russel's viper venom, ristocetin, and mixtures thereof. Generally, when clotting agents are used with the concentrated fibrinogen composition to form a fibrin glue, the clotting agent the concentrated fibrinogen composition are mixed immediately before application of the fibrin glue to an wound. The clotting agents can be added to or mixed with the concentrated fibrinogen composition to form fibrin glue. In one embodiment, the clotting agent can be present in a separate or second fluid which is mixed with the concentrated fibrinogen composition (i.e. a first fluid) immediately prior to the desired use time for the fibrin glue. In order to prevent premature formation of clotting, the first solution i.e. the concentrated fibrinogen composition, and the second solution containing the clotting agent can be maintained in separate containers until shortly before use. In one embodiment of the invention, the second solution can be provided by the wound itself in the form of wound fluids.

In another embodiment, the fibrin sealant can include calcium or magnesium. The addition of calcium or magnesium to the fibrinogen concentrate can increase the tensile and adhesion strengths of the resulting clot, presumably by acting, at least in part, as a co-factor of Factor XIII in crosslinking fibrin. In some cases, threshold concentrations of calcium magnesium can be required in the fibrin sealant to produce maximum effects (8.9 mM for the tensile strength, 3.6 mM for the adhesion strength—concentrations based on calcium or magnesium present as calcium chloride or magnesium chloride), suggesting that sufficient calcium or magnesium is needed to bind the free anionic components present in the fibrin fluid, e.g. citrate from sodium citrate, before its interaction with Factor XIII. Generally, calcium chloride or magnesium chloride concentrations in the fibrin sealant above 0.05 M do not have positive effects on the tensile strength of the resulting clot, and in some cases the tensile strength of the clot can be lessened. Without being limited by theory, it is believed that such a result is possibly due to an increase in ionic strength and partial precipitation of the fibrinogen, both adversely affecting the integrity of the clot. Generally, it is believed that any physiologically acceptable source of calcium or magnesium can be used including calcium or magnesium salts. In one embodiment, the calcium or magnesium can be present as calcium chloride (CaCl₂) or magnesium chloride (MgCl₂). In one embodiment, the calcium can be present as calcium chloride in the fibrinogen sealant at a concentration of from 1.8 nM to 100 nM calcium chloride. In another embodiment, the calcium can be present as calcium chloride in the fibrinogen sealant at a concentration of from 8.9 nM to 50 nM calcium chloride. In one embodiment, the magnesium can be present as magnesium chloride in the fibrinogen sealant at a concentration of from 1.8 nM to 100 nM magnesium chloride. In another embodiment, the calcium can be present as magnesium chloride in the fibrinogen sealant at a concentration of from 8.9 nM to 50 nM magnesium chloride.

When the fibrinogen sealants of the present invention are applied to wounds they help cement the gaps by adhering the tissue and stop the bleeding through the formation of clots. In one embodiment, the fibrinogen sealant can stop the bleeding of a subject in less than about 5 minutes. In another embodiment, the fibrinogen sealant can stop the bleeding of a subject in less than about 3 minutes. In yet a further embodiment, the fibrinogen sealant can stop the bleeding of a subject in less than about 1.5 minutes. Further, in another embodiment, the fibrinogen sealant can form a clot in vitro in less than about 5 minutes. In another embodiment, the fibrinogen sealant can form a clot in vitro in less than about 3 minutes. In yet another embodiment, the fibrinogen sealant can form a clot in vitro in less than about 1.5 minutes. In yet further embodiment, the fibrinogen sealant can form a clot in vitro in less than about 30 seconds.

### EXAMPLES

The following example illustrates preferred embodiments of the invention that are presently known. However, other embodiments can be practiced that are also within the scope of the present invention.

### Example 1

### Preparation of Platelet-Poor Plasma From Whole Blood

Blood is collected from healthy adult human donors by venipuncture into sodium citrate (Sigma Chemical Co., St. Louis, Mo.; final concentration 0.38 g/100 mL) according to the principles of the Declaration of Helsinki. The blood is centrifuged for 30 minutes at 1200 g to obtain platelet-poor plasma (PPP). The platelet-poor plasma can be used immediately for the preparation of fibrinogen concentrates or can be stored for use at a later time. When stored the PPP should be stored at −80° C.

### Example 2

### Preparation of Fibrinogen Concentrate From Pooled Human Plasma

Fibrinogen is precipitated from pooled human plasma by addition of protamine sulfate (Sigma Chemical Co.). The protamine sulfate is used to prepare a stock solution of 40 mg/mL. The protamine is then added to the plasma (final concentration=10 mg/ml), mixed, and then centrifuged at 1000 g for 5 min to sediment the precipitate. The plasma is then decanted, and the remaining precipitate is dissolved in 0.2 M sodium citrate (37° C., pH 7.4).

### Example 3

### Determination of Fibrinogen and Factor XIII Concentrations

A concentrated fibrinogen solution is prepared as in Example 2. The fibrinogen and Factor XIII concentrations are evaluated with an enzyme-linked immunosorbent assay (ELISA; AssayPro LLC, Brooklyn, N.Y.). The color intensity of the developed ELISA plates is measured with a Dynex MRX microplate reader (Dynex Technologies, Chantilly, Va.) and compared to a standard curve.

The fibrinogen concentration in the plasma is measured with the Clauss method, where plasma samples are clotted in the presence of excess thrombin in a CoaData 2000 Fibrintimer (Labor GmbH, Hamburg, Germany). The clotting times are recorded, and the fibrinogen concentration is calculated from a standard curve.

The amount of protamine bound with fibrinogen in the concentrate is determined by using ¹²⁵I-protamine. Two mg of protamine are labeled with ¹²⁵Iodine by utilizing IODO-GEN precoated tubes (Product 28601, Pierce, Rockford, Ill.) following their recommended protocol. In the final experiment, 1.0 mg ¹²⁵I-protamine is mixed with 99.0 mg unlabeled protamine and then added to 10 ml plasma. The resulting precipitate is washed three times with water, dissolved in 0.2 M sodium citrate and the amount of radioactivity associated with fibrinogen concentrate is measured by gamma counting.

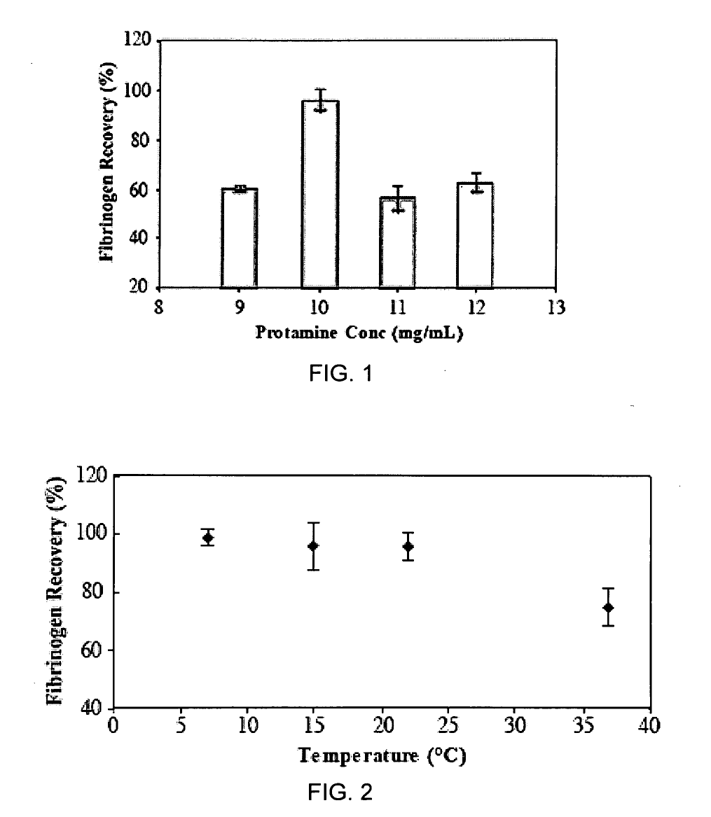

By varying the amount of protamine added to the plasma to achieve final protamine concentrations of 5 mg/mL to 15 mg/mL as guided by the literature various fibrinogen concentrations can be obtained. Maximum fibrinogen can be precipitated and recovered (96±4%, n=4) at a blood or plasma protamine concentration of 10 mg/mL (FIG. 1). Lower protamine concentrations precipitate less fibrinogen, and higher protamine concentrations can result in a precipitate of small dense aggregates that may be difficult to separate and may not readily dissolve.

The extraction efficiency of fibrinogen by using protamine precipitation is affected by temperature. The temperature-dependent nature of the fibrinogen precipitation can be investigated by adding protamine (10 mg/mL) to plasma samples at 37, 22, 15, and 7° C. Fibrinogen recovery is temperature independent at extraction temperatures of 22° C. and lower (FIG. 2) and is significantly better at 22° C. (96±4%, n=4 at 22° C.) than at 37° C. (75±6%, n=4).

The recovery of factor XIII in the fibrinogen concentrate when using plasma can reach a final concentration of 3.60±0.05 μg/mL, which is 47±0.6% (n=4) of the factor XIII in the initial plasma.

### Example 4

### Clottability of Precipitated Fibrinogen

The clottability of the recovered fibrinogen is evaluated as follows. A fibrinogen solution as prepared in Example 2 is prepared and used. To 1 mL of the fibrinogen solution 100 uL of bovine thrombin (Vital Products, Inc, Boynton Beach, Fla., 500 Units/mL) is added and the clot is allowed to stand for 30 minutes at 22° C. The clot is then centrifuged for 2 min at 3500 g and the supernatant removed. The amounts of fibrinogen present in the fibrinogen concentrate solution and in the clot supernatant are determined by ELISA, and the fibrinogen present in the clot is determined by difference.

To evaluate the incorporation of Factor XIII in the clot, the above process can be repeated with the addition of calcium chloride (Spectrum Quality Products, Inc., Gardena, Calif.). The amounts of fibrinogen and Factor XIII in the clot supernatant and in the concentrate can be measured with ELISA, and the amounts of fibrinogen and Factor XIII remaining in the clot can be determined by difference.

The fibrinogen in the concentrate polymerizes to form a clot, as described above. The amount of fibrinogen remaining in the clot is determined to be 98±0.9% (n=4) of the amount of fibrinogen in the original concentrate. No change in the clottability of the fibrinogen is observed when calcium chloride is added to the concentrate, and 30±1% of the factor XIII is associated with the clot (n=4).

### Example 5

### Effect of Heparin on Coagulation of Fibrinogen Concentrate

Heparin is used clinically in most procedures requiring anticoagulation. Heparin is evaluated for its effect on fibrinogen and Factor XIII harvesting and subsequent clotting of harvested fibrinogen. Blood is drawn into syringes containing porcine heparin (ESi Pharmaceuticals, Cherry Hill, N.J.; final concentration 2 U/mL) and centrifuged for 30 minutes at 1200 g to obtain PPP. Protamine was added to a known amount of plasma to bring the plasma concentrations to 10, 11, or 12 mg/mL. Fibrinogen concentrate was prepared as previously described above in Example 2. The amounts of fibrinogen and Factor XIII in the concentrate were measured with ELISA.

When the blood is collected into heparin, the maximum yield of fibrinogen occurs at a protamine concentration of 11 mg/mL in plasma (in contrast to 10 mg/mL when no heparin is present), precipitating 95±1% (n=4) of the fibrinogen in the plasma. At this protamine concentration, 31±3% (n=4) of the Factor XIII in the plasma is found in the concentrate. There are no observed changes in the clottability of fibrinogen when the heparin is present.

### Example 6

### Tensile Strength of Fibrin Clots

The tensile strength of fibrin clots is tested. A dog-bone shaped mold is machined in two halves from plexiglass and forms the shape of the clot. Stiff sponges are placed at the ends to allow the clot to form in/around them; the sponges, are held in the mold by bolts in removable plexiglass holders with O-ring seals. The clot diameter is 2 mm in the center of the narrow neck and 6.5 mm at the larger ends, the length is 31 mm, and the mold has a total volume of 1.5 mL. The narrow neck provided the weakest point where the clot would break; the force at which the clot breaks serves as an indication of its tensile strength.

Test samples are prepared by simultaneously emptying syringes of fibrinogen and thrombin into a common duct where the mixture entered the mold through the sponge on one end and exited through the sponge on the other end. Care is taken to avoid introduction of air during filling of the chamber. The sponges, with clot material penetrating their pores, provided a method to grip the clot firmly during testing. After the sample is given time to “cure,” (30 minutes unless various cure times were being tested), the plexiglass mold is dissembled, and the clot is transferred to an Instron Model 1120 Universal Testing Instrument (Instron Corp., Norwood, Mass., max load 500 g) where it is held on the ends via the sponge “grips”. A stress-strain curve is recorded while the sample is strained at 100 mm/min until it ruptured. The tensile strength is recorded as the maximum stress sustained.

### Example 7

### Adhesion Strength of Fibrin Clots

The adhesive strength of fibrin clots is tested. The adhesion strength of the fibrin glue is assessed by sandwiching the fibrin glue between two strips of aortic tissue and then pulling them apart, simulating the performance of the sealant bonding to tissue. Bovine aorta is prepared by slitting the aorta lengthwise and laying it flat. The aorta is then cut into smaller strips, each approximately 3 cm long and 1 cm wide. Since clots do not adhere to the endothelial lining, each strip is cut lengthwise between the adventitia and intima, yielding two thinner strips each with exposed media on one side. Sealant is applied (0.1 mL), covering an area of approximately 1 cm², to the exposed media as shown. An overlapping joint is formed (approximately one-third the length of each strip) and allowed to “cure” while held in place with a 100 g weight for 30 minutes at 22° C. The non-overlapping ends of the cured samples are clamped in an Instron Model 1120 Universal Testing Instrument (max load 500 g), and a stress-strain curve is recorded while the sample is strained at 100 mm/min until the overlapping (glued) joint failed. Adhesion strength is taken as the maximum stress sustained divided by the joint area (indicated by the glue still visible after the joint failed and measured with a digital caliper).

### Example 8

### Effect of Calcium on Tensile and Adhesion Strength

To assess whether the increase in tensile strength when calcium chloride was added to the fibrinogen concentrate (see the Results section) is due to Factor XIII, the tensile strengths of samples prepared from pure fibrinogen (Enzyme Research Laboratories, Swansea, Mid Glamorgan, UK) with and without added Factor XIII (Enzyme Research Laboratories, Swansea, Mid Glamorgan, UK, average functionality of 6200 Loewy units/mg) and calcium are measured. Samples are prepared from a 15 mg/mL pure fibrinogen concentrate (as prepared in Example 2) as follows:
1. fibrinogen alone
2. fibrinogen+calcium chloride (8.9 mM)
3. fibrinogen+factor XIII (10 μg/mL)
4. fibrinogen+factor XIII (10 μg/mL)+calcium chloride (8.9 mM)

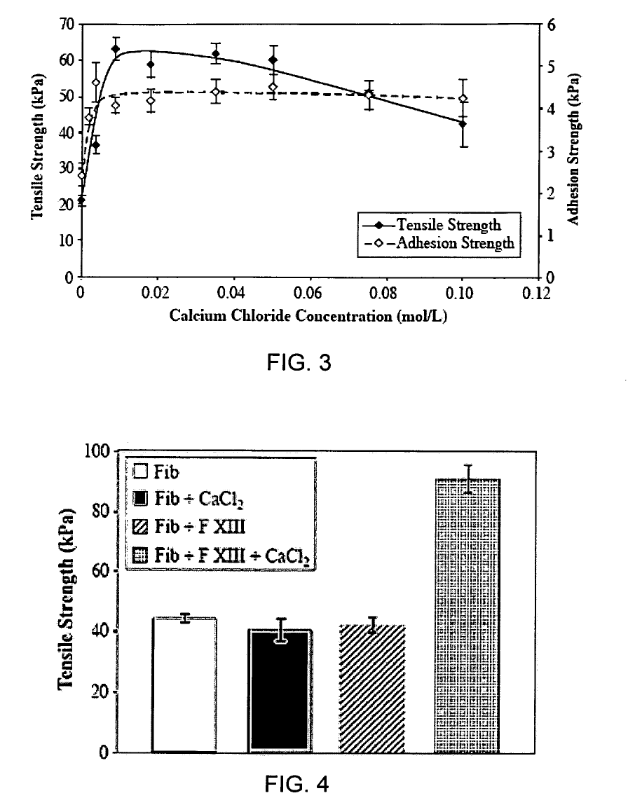

The effect of calcium on clot tensile strength and adhesion strength was investigated by adding calcium chloride (concentrations of 1.8 to 100 mM) to 15 mg/mL fibrinogen concentrate. Maximum tensile strength was achieved with calcium concentrations in the range of 8.9-50 mM, and maximum adhesion strength was obtained with calcium concentrations of 3.6-100 mM (FIG. 3).

Clots were prepared from pure fibrinogen with and without calcium and Factor XIII addition as described above. When Factor XIII and calcium were added together, the tensile strength of the clots increased approximately 50 kPa (FIG. 4), which is similar to the increase of 65 kPa seen in the tensile strength of sealant when the calcium concentration was increased from 0 to 8.9 mM (FIG. 3).

### Example 9

### Effect of Cure Time on Tensile Strength

The effect of cure time on tensile strength and adhesion strength is evaluated by allowing the molded clots and the glued aortic strips (described in Example 7) to cure for 1, 5, 10, 15, 30, and 60 minutes at 22° C. Samples are prepared from a 15 mg/mL fibrinogen concentrate with and without calcium chloride added (8.9 mM).

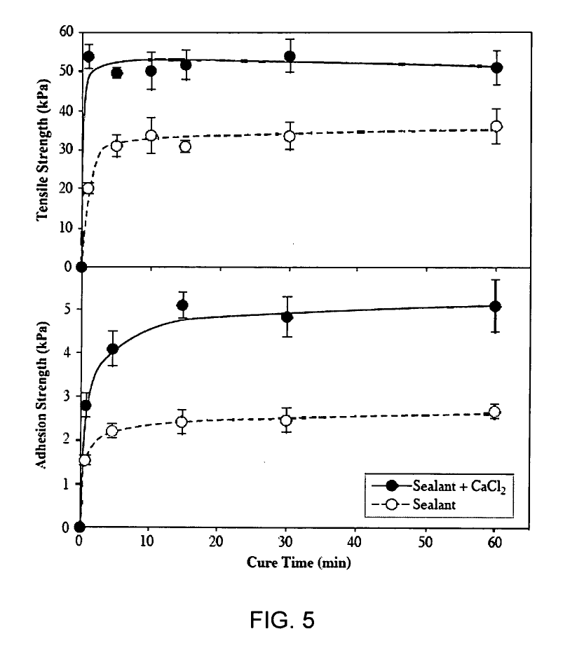

For clots cured for various times, maximum tensile strength was reached in 1 minute (the shortest time that could be measured) with calcium added and about 5 minutes without calcium added (FIG. 5). The maximum adhesion strength with calcium present was approximately twice the adhesion strength without calcium but required a longer cure time to achieve (15 minutes versus 5 minutes).

### Example 10

### Effect of Fibrinogen Concentration on Tensile Strength

To evaluate the effect of fibrinogen concentration on tensile strength and adhesion strength, samples are prepared with fibrinogen concentrations of 15, 30, 45, and 60 mg/mL, with and without calcium chloride added (final concentration 8.9 mM). Controls of pooled human plasma (fibrinogen concentration ˜3 mg/mL), pure fibrinogen (15 mg/mL), and Tisseel (average fibrinogen concentration ˜95 mg/mL) are used. Molded clots and adhesive joints were cured for 30 min.

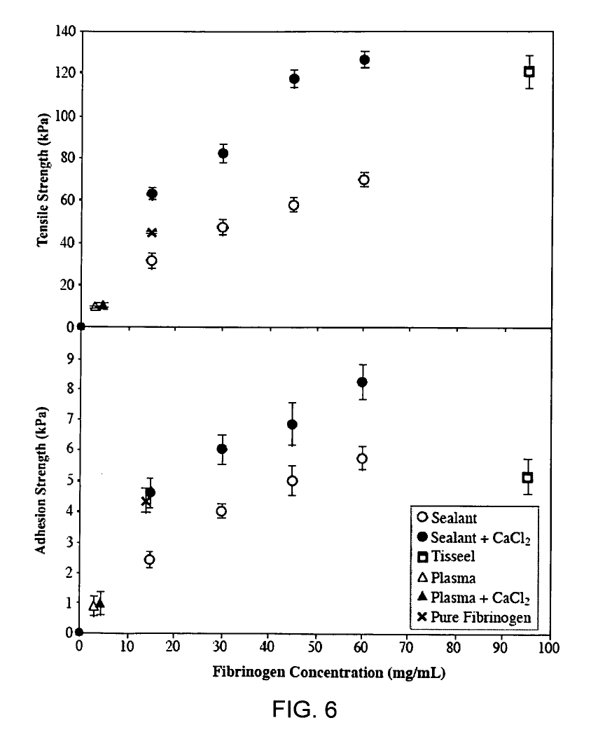

Tensile and adhesion strengths were found to increase approximately linearly with increasing fibrinogen concentration (FIG. 6). The adhesion strength of the sample prepared from plasma fell near the curve with the samples prepared from protamine-fibrinogen concentrate. The tensile strength of the 15 mg/mL pure fibrinogen sample was significantly greater than that of the 15 mg/mL protamine-fibrinogen sample (p<0.05). It appeared that the presence of protamine in the fibrinogen concentrate lowered the adhesion strength of the resulting glue as compared with glue formed with concentrated fibrinogen.

At each fibrinogen concentration, the addition of calcium chloride significantly increased the tensile strength (p<0.05) and adhesion strength (p<0.05) compared with the fibrinogen concentrate with no calcium added. No change in tensile strength or adhesion strength was observed when calcium chloride was added to pure fibrinogen, presumably because there was no Factor XIII present in the pure fibrinogen concentrate. Also, no change in tensile or adhesion strength was observed when calcium chloride was added to citrated plasma. This may have been because either 1) some free calcium was still present in the citrated plasma, thus enabling the Factor XIII action, even when no calcium chloride was added, or 2) the Factor XIII concentration in the plasma was low (normally 10 μg/mL in plasma compared with 20, 50, 70, and 95 μg/mL in the protamine-fibrinogen concentrates).

Tisseel exhibited tensile strength similar to that of sealant made from protamine-fibrinogen concentrate (45-60 mg/mL fibrinogen) with calcium added and adhesion strength similar to that of sealant made from protamine-fibrinogen concentrate (45-60 mg/mL fibrinogen) with no calcium added. The Tisseel adhesion strength was significantly less than that of the sealant glue formed with 30, 45 and 60 mg/mL fibrinogen concentrates with calcium chloride added (p<0.05).

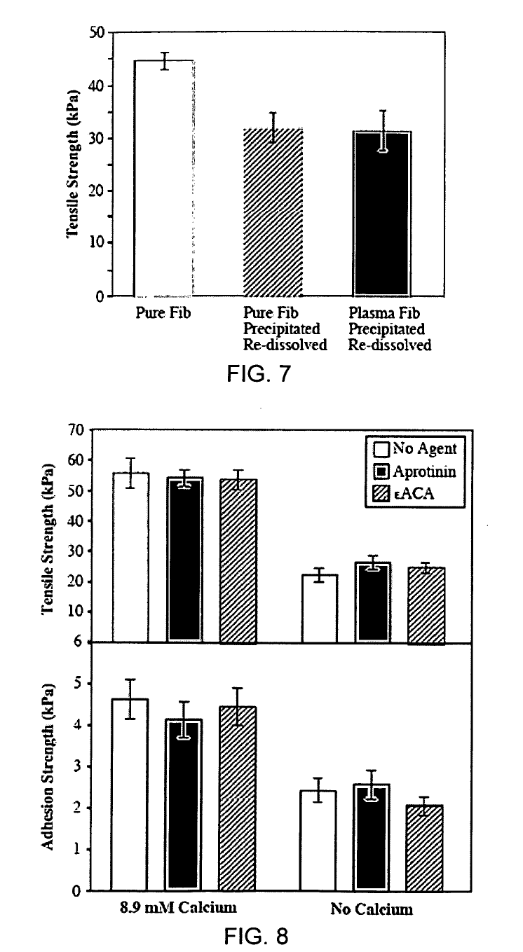

The tensile and adhesion strengths of the 15 mg/mL pure fibrinogen sample were significantly higher than those of the 15 mg/mL protamine-fibrinogen sample (p<0.05). The major difference between these two preparations is the precipitation with protamine in one case. To test the hypothesis that the addition of protamine adversely affected the tensile strength, a fibrinogen concentrate (15 mg/mL) was prepared by protamine precipitation of pure fibrinogen to compare with a 15 mg/mL pure fibrinogen concentrate prepared without precipitation (fibrinogen concentrations were confirmed in both samples). The tensile strength of the protamine-precipitated pure fibrinogen was significantly lower (p<0.05) than that of the pure fibrinogen (FIG. 7), presumably because of the presence of protamine in the concentrate.

### Example 11

### Effect of Fibrinolytic Inhibitors on Tensile Strength

Because enzymes responsible for fibrinolysis in the plasma may affect the clot tensile and adhesion strengths, the effect of the presence of fibrinolytic inhibitors on tensile and adhesion strengths was investigated. Samples were prepared from a 15 mg/mL fibrinogen concentrate with or without calcium chloride added (8.9 mM). In some samples, Aprotinin (Trasylol Injection, Bayer Corp., West Haven, Conn.) was added to the fibrinogen concentrate (final concentration=3000 KIU/mL). In other samples, ε-Aminocaproic acid (Sigma Chemical Co.) was added to the fibrinogen concentrate (final concentration=10 mg/mL). There were no significant changes in tensile strength or adhesion strength upon addition of the antifibrinolytic agents (FIG. 8).

### Example 12

### Preparation of Autologous Fibrin Glue From Whole Blood

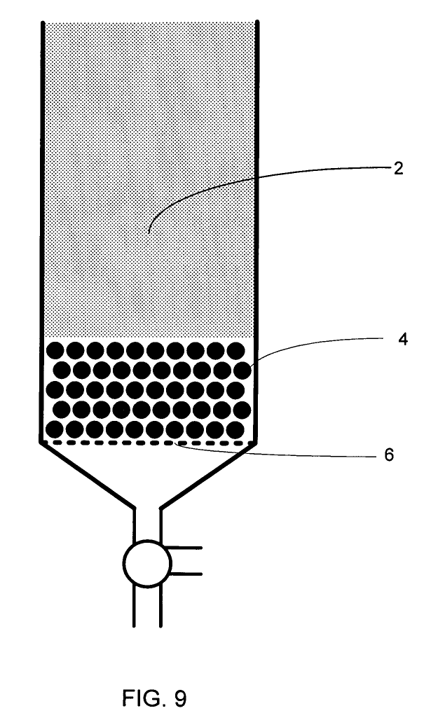

Citrated blood (20 ml) is collected using a blue-top vacutainer system and transferred to a 30 ml syringe predispensed with 200 mg protamine (4.0 ml from a 50 mg/ml solution), mixed gently for 5 min, and the mixed solution of protamine and blood 2 is poured into a specially-designed tube shown in FIG. 9. The precipitated fibrinogen is captured on a glass-bead 4 (0.1-mm diameter beads in a 1-cm column retained by a nylon mesh filter) as the blood passes through the filter 6. Once all of the blood is drained, the filter is rinsed with three 15-ml aliquots of saline (0.15 M NaCl) to remove nonadherent cells/proteins. After the third rinse, any saline remaining in the tube is drained, the stopcock is closed, and 2.0 ml 0.2M sodium citrate is added. After thorough mixing with a Pasteur pipette, the fluid is drained into a 3-ml syringe as the fibrinogen concentrate. When the fibrinogen concentrate is mixed with a solution of thrombin (500 units/ml of fibrinogen concentrate in 2M CaCl₂; 1:4 vol/vol of concentrate) a viscous fibrin gel forms instantaneously and serves as fibrin sealant. The time from adding the blood to the mixing chamber to the recovery of concentrate is usually less than 15 min. The fibrinogen concentrate prepared from whole blood exhibits physicochemical characteristics similar to the commercially available fibrin glue Tisseel V (Baxter Healthcare, Calif.)

It is to be understood that the above-referenced arrangements are only illustrative of the application for the principles of the present invention. Numerous modifications and alternative arrangements can be devised without departing from the spirit and scope of the present invention while the present invention has been shown in the drawings and fully described above with particularity and detail in connection with what is presently deemed to be the most practical and preferred embodiments(s) of the invention, it will be apparent to those of ordinary skill in the art that numerous modifications can be made without departing from the principles and concepts of the invention as set forth in the claims.

What is claimed is:

1. A method of making and clotting a concentrated fibrinogen composition, comprising:
adding a sufficient amount of a cationic agent to a fibrinogen containing fluid to cause the fibrinogen to form a fibrinogen precipitate;
collecting the fibrinogen precipitate;
after collecting, suspending or solubilizing the fibrinogen precipitate in a liquid vehicle to form a concentrated fibrinogen composition; and
clotting the concentrated fibrinogen composition.

2. The method of claim 1, wherein the concentrated fibrinogen composition has a concentration which is at least twice the fibrinogen concentration in the fibrinogen containing fluid.

3. The method of claim 1, wherein the collecting is performed by gravity settling, centrifugation, filtration, or combinations thereof.

4. The method of claim 3, wherein the collecting is performed by filtration.

5. The method of claim 4, wherein the collecting is performed using a portable filtration device.

6. The method of claim 3, wherein the collecting is performed by centrifugation.

7. The method of claim 1, wherein the liquid vehicle includes a member selected from the group consisting of sodium citrate, sodium hydroxide, potassium hydroxide, heparin, heparan sulfate, and mixtures thereof.

8. The method of claim 1, wherein the liquid vehicle includes sodium citrate.

9. The method of claim 1, wherein the fibrinogen containing fluid is whole blood.

10. The method of claim 1, wherein the cationic agent is selected from the group consisting of protamine, polylysine, polyallylamine, histones, and mixtures thereof.

11. The method of claim 10, wherein the cationic agent is protamine.

12. The method of claim 1, wherein the fibrinogen containing fluid is plasma.

13. The method of claim 1, wherein the collecting is performed by centrifugation and the liquid vehicle includes sodium citrate.

14. The method of claim 1, wherein the fibrinogen is present in the concentrated fibrinogen composition at a concentration of 10 mg/ml to 200 mg/ml.

15. The method of claim 1, wherein the fibrinogen is present in the concentrated fibrinogen composition at a concentration of 20 mg/ml to 100 mg/ml.

16. The method of claim 1, wherein the fibrinogen is present in the concentrated fibrinogen composition at a concentration of from 20 mg/ml to 60 mg/ml.

17. The method of claim 1, wherein the fibrinogen is present in the concentrated fibrinogen composition at a concentration of at least about 15 mg/ml of fibrinogen.

18. The method of claim 1, wherein the concentrated fibrinogen composition further includes at least one clotting factor selected from the group consisting of Factor II, Factor IX, Factor X, and Factor XIII.

19. The method of claim 1, wherein the concentrated fibrinogen composition further includes at least two clotting factors selected from the group consisting of Factor IX, Factor X, Factor XIII, and Factor II.

20. The method of claim 1, wherein the concentrated fibrinogen composition further includes at least three clotting factors selected from the group of Factor II, Factor IX, Factor X, and Factor XIII.

21. The method of claim 1, wherein the concentrated fibrinogen composition further includes each of the clotting factors Factor II, Factor IX, Factor X, and Factor XIII.

22. The method of claim 1, wherein the clotting step includes mixing a clotting agent with the concentrated fibrinogen composition.

23. The method of claim 22, wherein the clotting agent is selected from the group consisting of calcium salts, magnesium salts, thromboplastin, actin, thrombin, collagen, platelet suspension, precipitated or denatured proteins, complex carbohydrates, silica, zinc, diatomaceous earth, kaolin, Russel's viper venom, ristocetin, and mixtures thereof.

24. The method of claim 22, wherein the clotting agent is a calcium salt.

25. A method of treating wounds, comprising:
a) adding a sufficient amount of a cationic agent to a fibrinogen containing fluid to cause the fibrinogen to form a fibrinogen precipitate;
b) collecting the fibrinogen precipitate;
c) after collecting, suspending or solubilizing the fibrinogen precipitate in a liquid vehicle to form a concentrated fibrinogen composition;
d) mixing the concentrated fibrinogen composition with a clotting agent to form a fibrin sealant, and
e) applying an amount of the fibrin sealant to a wound, thereby forming a clot.

26. The method of claim 25, wherein the wound is a surgically created incision.

27. The method of claim 25, wherein the wound is a result of an injury.

28. The method of claim 27, wherein the injury is an ulcer, broken bone, or torn tissue.

29. The method of claim 25, wherein at a site of the wound, there is active bleeding at a body surface.

30. The method of claim 25, wherein the clotting agent is selected from the group consisting of calcium salts, magnesium salts, thromboplastin, actin, thrombin, collagen, platelet suspension, precipitated or denatured proteins, complex carbohydrates, silica, zinc, diatomaceous earth, kaolin, Russel's viper venom, ristocetin, and mixtures thereof.

31. The method of claim 25, wherein thrombin is present in the fibrin sealant at a concentration of 50 units/ml to 500 units/ml of the fibrin sealant

32. The method of claim 25, wherein the concentrated fibrinogen composition includes at least one clotting factor selected from the group Factor II, Factor IX, Factor X, and Factor XIII.

33. The method of claim 25, wherein the clotting agent includes calcium, magnesium, or a mixture thereof.

34. The method of claim 33, wherein the clotting agent includes calcium, and is present in the fibrinogen sealant as calcium chloride at a concentration from 1.8 nM to 100 nM.

35. The method of claim 33, wherein the clotting agent includes calcium, and is present in the fibrinogen sealant as calcium chloride at a concentration from 8.9 nM to 50 nM.

36. The method of claim 33, wherein the clotting agent includes magnesium, and is present in the fibrinogen sealant as magnesium chloride at a concentration from 1.8 nM to 100 nM.

37. The method of claim 33, wherein the clotting agent includes magnesium, and is present in the fibrinogen sealant as magnesium chloride at a concentration from 8.9 nM to 50 nM.

38. The method of claim 25, wherein the fibrinogen sealant forms the clot in less than 5 minutes.

39. The method of claim 25, wherein the fibrinogen sealant forms the clot in less than about 3 minutes.

40. The method of claim 25, wherein the fibrinogen sealant forms the clot in less than about 1.5 minutes.

41. The method of claim 25, wherein the fibrinogen sealant forms the clot in less than about 30 seconds.

42. A method of making a concentrated fibrinogen composition from blood, comprising:
adding a sufficient amount of a cationic agent to blood so as to cause fibrinogen present in the whole blood to form a fibrinogen precipitate;
collecting the fibrinogen precipitate; and
after collecting, suspending or solubilizing the fibrinogen precipitate in a liquid vehicle to form a concentrated fibrinogen composition.

43. The method of claim 42, wherein the concentrated fibrinogen composition has a fibrinogen concentration which is at least twice the concentration of fibrinogen in the fibrinogen containing fluid.

44. The method of claim 42, wherein the method further includes the step of clotting the concentrated fibrinogen composition.

45. The method of claim 42, wherein the collecting is performed by gravity settling, centrifugation, filtration, or combinations thereof.

46. The method of claim 45, wherein the collecting is performed by filtration.

47. The method of claim 42, wherein the collecting is performed by centrifugation.

48. The method of claim 42, wherein the liquid vehicle includes a member selected from the group consisting of sodium citrate, sodium hydroxide, potassium hydroxide, heparin, heparan sulfate, and mixtures thereof.

49. The method of claim 42, wherein the fibrinogen is present in the concentrated fibrinogen composition at a concentration of 10 mg/ml to 200 mg/ml.

50. The method of claim 42, wherein the fibrinogen is present in the concentrated fibrinogen composition at a concentration of 20 mg/ml to 100 mg/ml.

51. The method of claim 42, wherein the fibrinogen is present in the concentrated fibrinogen composition at a concentration of from 20 mg/ml to 60 mg/ml.

52. The method of claim 42, wherein the concentrated fibrinogen composition further includes at least one clotting factor selected from the group consisting of Factor II, Factor IX, Factor X, and Factor XIII.

53. The method of claim 42, wherein the at least one clotting factor is present in the concentrated fibrinogen composition at concentration which is at least 25% the concentration of the at least one clotting factor in whole blood.

54. The method of claim 42, wherein the at least one clotting factor is present in the concentrated fibrinogen composition at concentration which is at least 50% the concentration of the at least one clotting factor in whole blood.

55. The method of claim 42, wherein the at least one clotting factor is present in the concentrated fibrinogen composition at concentration which is at least 75% the concentration of the at least one clotting factor in whole blood.

56. The method of claim 42, wherein the concentrated fibrinogen composition further includes at least two clotting factors selected from the group consisting of Factor II, Factor IX, Factor X, and Factor XIII.

57. The method of claim 42, wherein the concentrated fibrinogen composition further includes at least three clotting factors selected from the group consisting of Factor II, Factor IX, Factor X, and Factor XIII.

58. The method of claim 42, wherein the cationic agent is selected from the group consisting of protamine, polylysine, polyallylamine, histones, and mixtures thereof.

59. A method of making and using an autologous fibrin glue, comprising:
collecting a fibrinogen containing fluid from a subject;
adding a sufficient amount of a cationic agent to the fibrinogen containing fluid sample to cause the fibrinogen to form a fibrinogen precipitate;
collecting the fibrinogen precipitate;
suspending or solubilizing the fibrinogen precipitate in a liquid vehicle to form a concentrated fibrinogen composition; and
applying the concentrated fibrinogen composition to a wound of the subject to form a fibrin glue, wherein the fibrin glue forms a clot.

60. The method of claim 59, wherein the collecting is performed by gravity settling, centrifugation, filtration, or combinations thereof.

61. The method of claim 59, wherein the liquid vehicle includes a member selected from the group consisting of sodium citrate, sodium hydroxide, potassium hydroxide, heparin, heparan sulfate, and mixtures thereof.

62. The method of claim 59, wherein the fibrinogen containing fluid is whole blood.

63. The method of claim 59, wherein the fibrinogen containing fluid is plasma.

64. The method of claim 59, wherein the fibrinogen is present in the concentrated fibrinogen composition at a concentration of 10 mg/ml to 200 mg/ml.

65. The method of claim 59, wherein the fibrinogen is present in the concentrated fibrinogen composition at a concentration of 20 mg/ml to 100 mg/ml.

66. The method of claim 59, wherein the fibrinogen is present in the concentrated fibrinogen composition at a concentration of from 20 mg/ml to 60 mg/ml.

67. The method of claim 59, wherein the concentrated fibrinogen composition further includes at least one clotting factor selected from the group consisting of Factor II, Factor IX, Factor X, and Factor XIII.

68. The method of claim 59, wherein the concentrated fibrinogen composition further includes at least two clotting factors selected from the group consisting of Factor II, Factor IX, Factor X, and Factor XIII.

69. The method of claim 59, wherein the concentrated fibrinogen composition further includes at least three clotting factors selected from the group consisting of Factor II, Factor IX, Factor X, and Factor XIII.

70. The method of claim 59, wherein the concentrated fibrinogen composition is applied to the wound with a clotting agent which acts to enhance the speed of formation of the fibrin glue on the wound.

71. The method of claim 70, wherein the clotting agent is selected from the group consisting of calcium salts, magnesium salts, thromboplastin, actin, thrombin, collagen, platelet suspension, precipitated or denatured proteins, complex carbohydrates, silica, zinc, diatomaceous earth, kaolin, Russel's viper venom, ristocetin, and mixtures thereof.

72. The method of claim 70, wherein the clotting agent and the concentrated fibrinogen composition are mixed immediately before or during the applying step.

73. The method of claim 59, wherein the concentrated fibrinogen composition forms the fibrin glue on the wound due to its interaction with body fluid present at the site of the wound.

74. The method of claim 59, wherein the cationic agent is selected from the group consisting of protamine, polylysine, polyallylamine, histones, and mixtures thereof.

75. A system for making a fibrin glue, comprising:
a first component, said first component being a fluid including 10 mg/ml to 200 mg/ml fibrinogen and at least one clotting factor selected from the group consisting of Factor II, Factor IX, Factor X, and Factor XIII; and
a second component including a clotting agent for said fibrinogen, wherein when said first component and second component are contacted, a fibrin glue is formed.

76. The system of claim 75, wherein the clotting factor is Factor X.

77. The system of claim 76, wherein Factor X is present in the first component at a concentration which is at 25% the concentration of Factor X in normal blood.

78. The system of claim 75, wherein the clotting factor is Factor II.

79. The system of claim 78, wherein Factor II is present in the first component at a concentration which is at 25% the concentration of Factor II in normal blood.

80. The system of claim 75, wherein the clotting factor is Factor XIII.

81. The system of claim 80, wherein Factor XIII is present in the first component at a concentration which is at 25% the concentration of Factor XIII in normal blood.

82. The system of claim 75, wherein at least two of the clotting factors are present in the first component.

83. The system of claim 75, wherein at least three of the clotting factors are present in the first component.

84. The system of claim 75, wherein each of the clotting factors Factor II, Factor IX, Factor X, and Factor XIII are present in the first fluid.

85. The system of claim 75, wherein the fibrinogen is present in the first component at a concentration of 10 mg/ml to 100 mg/ml.

86. The system of claim 75, wherein the fibrinogen is present in the first component at a concentration of 20 mg/ml to 60 mg/ml.

87. The system of claim 75, wherein the second component is provided by a wound.

88. The system of claim 75, wherein the system is configured such that the first component and the second component are present in separate containers.

89. The system of claim 75, wherein the clotting agent is selected from the group consisting of calcium salts, magnesium salts, thromboplastin, actin, thrombin, collagen, platelet suspension, precipitated or denatured proteins, complex carbohydrates, silica, zinc, diatomaceous earth, kaolin, Russel's viper venom, ristocetin, and mixtures thereof.

90. The system of claim 75, wherein the first fluid is manufactured by:
adding a sufficient amount of protamine to a fibrinogen containing fluid to cause the fibrinogen to form a fibrinogen precipitate;
collecting the fibrinogen precipitate; and
suspending or solubilizing the fibrinogen precipitate in a liquid vehicle to form the first fluid.

91. A method of making a concentrated fibrinogen composition, comprising:
adding a sufficient amount of protamine to a fibrinogen containing fluid to cause the fibrinogen to form a fibrinogen precipitate;
collecting the fibrinogen precipitate by centrifugation; and
after collecting, suspending or solubilizing the fibrinogen precipitate in a sodium citrate containing liquid vehicle to form a concentrated fibrinogen composition; wherein the concentration of the fibrinogen in the concentrated fibrinogen composition is at least twice the concentration of fibrinogen in the fibrinogen containing fluid.

92. The method of claim 91, wherein the fibrinogen containing fluid is whole blood.

93. The method of claim 91, wherein the fibrinogen containing fluid is plasma.

94. The method of claim 91, wherein the fibrinogen is present in the concentrated fibrinogen composition at a concentration of 10 mg/ml to 200 mg/ml.

95. The method of claim 91, wherein the fibrinogen is present in the concentrated fibrinogen composition at a concentration of from 20 mg/ml to 60 mg/ml.

96. The method of claim 91, wherein the concentrated fibrinogen composition further includes at least one clotting factor selected from the group consisting of Factor II, Factor IX, Factor X, and Factor XIII.

97. A system for making a fibrin glue, comprising:
a fluid including 10 mg/ml to 200 mg/ml fibrinogen and at least one clotting factor selected from the group consisting of Factor II, Factor IX, Factor X, and Factor XIII, wherein when applied to a wound, a fibrin glue is formed.
